(19) United States
(12) Patent Application Publication
(10) Pub. No.: US 2007/0293702 A1
(43) Pub. Date: Dec. 20, 2007

(54) Novel crystalline forms of armodafinil and preparation thereof

(76) Inventors: Viviana Braude, Kadima (IL); Rinat Moshkovits-Kaptsan, Raanana (IL); Sigalit Levi, Modi'in (IL); Judith Aronhime, Rehovot (IL)

Correspondence Address: KENYON & KENYON LLP, ONE BROADWAY, NEW YORK, NY, 10004, US

(21) Appl. No.: 11/709,906

(22) Filed: Feb. 21, 2007

Related U.S. Application Data
(60) Provisional application No. 60/775,572, filed on Feb. 21, 2006.
(60) Provisional application No. 60/858,758, filed on Nov. 13, 2006.

(51) Int. Cl. C07C 233/05

(52) U.S. Cl. 564/162

(57) ABSTRACT

The invention encompasses crystalline forms of armodafinil, processes for preparing the crystalline forms, and pharmaceutical formulation

### RELATED APPLICATIONS

The application claims the benefit of U.S. provisional application Nos. 60/775,572 filed Feb. 21, 2006 and 60/858,758 filed Nov. 13, 2006, hereby incorporated by reference.

### FIELD OF THE INVENTION

The invention encompasses crystalline armodafinil forms, processes for preparing the crystalline forms, and pharmaceutical formulations thereof.

### BACKGROUND OF THE INVENTION

Modafinil is currently marketed by Cephalon, Inc. under the trade name Provigil® as a racemic mixture of its R and S enantiomers. Provigil® is indicated for the treatment of excessive sleepiness associated with narcolepsy, shift work sleep disorder (SWSD), and obstructive sleep apnea/hypopnea syndrome (OSA/HS).

Studies have shown that while both enantiomers of modafinil are pharmacologically active, the S enantiomer is eliminated from the body three times faster than the R enantiomer. Prisinzano et al., Tetrahedron: Asymmetry, vol. 5 1053-1058 (2004). It is, therefore, preferable to develop pharmaceutical compositions of the R enantiomer of modafinil, as opposed to its racemic mixture.

The R enantiomer of modafinil is known as armodafinil and has the chemical name 2-[(R)-(diphenylmethyl)sulfinyl]acetamide. The molecular weight of armodafinil is 273.34 and it has the following chemical structure: 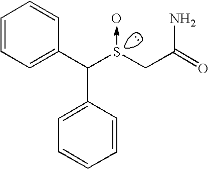
Armodafinil is commercially available as Nuvigil™.

Armodafinil and a method of its preparation were first disclosed in U.S. Pat. No. 4,927,855 (“'855 patent”) and EP patent No. 0233106, both of which were originally assigned to Lafon Laboratories. The '855 patent describes the synthesis of armodafinil by the following general scheme: 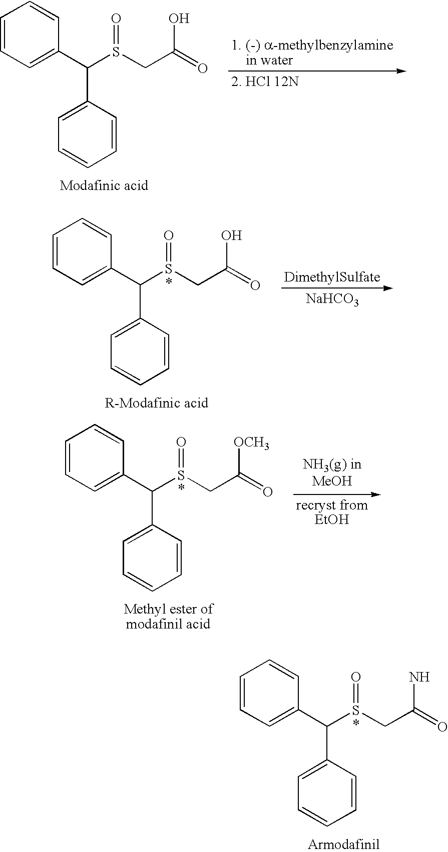
See '855 patent, col. 2, 11. 16-53.

Armodafinil can exist in several crystalline forms, some of which are disclosed in WO 2005/023198 (“WO '198”), WO 2005/077894 (“WO '894”), and WO 2004/060858 (“WO '858”). WO '858 discloses armodafinil Forms I-IV and an amorphous form, as well as dimethylcarbonate, acetic acid and acetonitrile solvates of armodafinil. WO '858, pages 10-12. WO '894 discloses armodafinil Forms III-V, as well as chloroform, chlorobenzene, and acetic acid solvates of armodafinil. WO '894, pages 3, 8. WO '198 discloses additional solvated forms of armodafinil, such as acetonitrile, ethanol, benzyl alcohol, and isopropanol solvates. WO '198, page 9. Form I is characterized by d-spacing values at about: 13.40, 8.54, 6.34, 5.01, 4.68, 4.62, 4.44, 4.27, 4.20, 4.15, 4.02, 3.98, 3.90, 3.80, and 3.43 (angstroms). Form IV is characterized by d-spacing values at about: 13.88, 12.38, 10.27, 8.58, 7.34, 6.16, 5.66, 5.12, 5.00, 4.64, 4.48, 4.26, 4.18, 4.09, 3.82, 3.66, 3.53, 3.42, 3.28, and 3.20 (angstroms).

The occurrence of different crystal forms (polymorphism) is a property of some molecules and molecular complexes. A single molecule, like armodafinil, may give rise to a variety of solids having distinct physical properties such as melting point, X-ray diffraction pattern, infrared absorption fingerprint, and NMR spectrum. The differences in the physical properties of polymorphs result from the orientation and intermolecular interactions of adjacent molecules (complexes) in the bulk solid. Accordingly, polymorphs are distinct solids sharing the same molecular formula yet having distinct advantageous and/or disadvantageous physical properties compared to other forms in the polymorph family. One of the most important physical properties of pharmaceutical polymorphs is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. On the other hand, where the effectiveness of a drug correlates with peak bloodstream levels of the drug, a property shared by statin drugs, and provided the drug is rapidly absorbed by the GI system, then a more rapidly dissolving form is likely to exhibit increased effectiveness over a comparable amount of a more slowly dissolving form.

The discovery of new crystalline polymorphic forms of a drug enlarges the repertoire of materials with which a formulation scientist can design a pharmaceutical dosage form of a drug with a targeted release profile or other desired characteristics. Therefore, there is a need to find more crystalline forms of armodafinil.

### SUMMARY OF THE INVENTION

In one embodiment the present invention provides a crystalline form of armodafinil characterized by a powder XRD pattern having peaks at about 6.5, 10.3, 17.9, 19.5 and 21.8±0.2 degrees 2-theta.

In one embodiment the present invention provides a process of preparing the above crystalline form comprising crystallizing the crystalline form from acetonitrile.

A crystalline form of armodafinil characterized by a powder XRD pattern having peaks at about 7.0, 9.3, 12.2, 14.4 and 16.2±0.2 degrees 2-theta.

In one embodiment the present invention provides a process of preparing the above crystalline form comprising crystallizing the crystalline form from acetic acid.

In one embodiment the present invention provides a process for preparing crystalline armodafinil acetic acid solvate comprising drying crystalline armodafinil Form B.

In one embodiment the present invention provides a crystalline form of armodafinil characterized by a powder XRD pattern having peaks at about 5.4, 9.1, 10.3 and 10.8±0.2 degrees 2-theta.

In one embodiment the present invention provides a process of preparing the above crystalline form comprising crystallizing the crystalline form from dioxane.

In one embodiment the present invention provides a crystalline form of armodafinil characterized by a powder XRD pattern having peaks at about 9.4, 12.5, 14.5 and 18.6±0.2 degrees 2-theta.

In one embodiment the present invention provides a process of preparing the above crystalline form comprising crystallizing the crystalline form from dimethyl carbonate.

In one embodiment the present invention provides a crystalline form of armodafinil characterized by a powder XRD pattern having peaks at about 7.1, 9.1, 12.2 and 16.3±0.2 degrees 2-theta.

In one embodiment the present invention provides a process of preparing the above crystalline form comprising crystallizing the crystalline form in methylethylketone.

In one embodiment the present invention provides a process for preparing armodafinil Form I comprising drying or humidifying any of the preceding crystalline forms.

In one embodiment the present invention provides a process for preparing armodafinil Form I comprising drying armodafinil Form C.

In one embodiment the present invention provides a process for preparing armodafinil Form I comprising drying armodafinil Form D.

In one embodiment the present invention provides a process for preparing armodafinil Form I comprising exposing armodafinil Form A to 100% relative humidity at a temperature of about 20° C. to about 40° C.

In one embodiment the present invention provides a process for preparing armodafinil Form I comprising drying armodafinil Form IV.

In one embodiment the present invention provides a process for preparing armodafinil Form I comprising submitting armodafinil Form IV to a pressure of about 2 tons to about 10 tons to obtain armodafinil Form I. The process can be stopped prematurely to obtain a mixture.

In one embodiment the present invention provides a crystalline form of armodafinil characterized by a weight loss that is 0.3% at the range of about 25° C. to about 100° C. as measured by TGA.

In one embodiment the present invention provides a process for preparing armodafinil Form I comprising grinding armodafinil Form IV.

In one embodiment the present invention provides a Crystalline armodafinil THF solvate.

In one embodiment the present invention provides a crystalline armodafinil THF solvate characterized by having PXRD peaks at 7.3, 9.3, 10.4, 12.3, 14.4, 17.8, 21.6, 23.6, and 24.7±0.2 degrees 2-theta.

In one embodiment the present invention provides a process for preparing armodafinil THF solvate comprising crystallizing the crystalline form from THF.

In one embodiment the present invention provides a crystalline armodafinil methyl acetate solvate.

In one embodiment the present invention provides a Crystalline armodafinil methyl acetate solvate characterized by having PXRD peaks at 7.3, 9.3, 10.4, 12.3, 14.4, 17.8, 21.6, 23.6, and 24.7±0.2 degrees 2-theta.

In one embodiment the present invention provides a process of preparing the above crystalline form comprising crystallizing the crystalline form from methyl acetate.

In one embodiment the present invention provides a process for preparing armodafinil Form IV comprising drying armodafinil THF solvate.

In one embodiment the present invention provides a process for preparing armodafinil Form IV comprising drying armodafinil Form E.

In one embodiment the present invention provides a process for preparing armodafinil Form IV comprising drying armodafinil methyl acetate solvate.

In one embodiment the present invention provides a Crystalline Armodafinil hydrate.

In one embodiment the present invention provides a Crystalline Armodafinil hemihydrate.

In one embodiment the present invention provides a Crystalline Armodafinil hemihydrate characterized by PXRD peaks at 6.8, 10.5, 13.5, 14.2, 19.2, 20.2, 21.1, 22.5, 23.7, and 26.2±0.2 degrees 2-theta.

In one embodiment the present invention provides a process for preparing armodafinil hemihydrate, comprising exposing armodafinil Form A to humidity.

In one embodiment the present invention provides a crystalline armodafinil monohydrate.

In one embodiment the present invention provides a process for preparing armodafinil monohydrate comprising exposing armodafinil Form A to humidity.

In one embodiment the present invention provides a pharmaceutical composition comprising a therapeutically effective amount of at least one of armodafinil Forms A, B, C, D, or E and at least one pharmaceutically acceptable excipient.

In one embodiment the present invention provides a process for preparing a pharmaceutical composition of armodafinil comprising combining at least one of armodafinil Forms A, B, C, D, or E and at least one pharmaceutically acceptable excipient.

In one embodiment the present invention provides a the use of the above pharmaceutical composition in the manufacture of a medicament for treatment of excessive sleepiness.

### BRIEF DESCRIPTION OF THE FIGURES

FIG. 1 illustrates the powder XRD pattern of armodafinil Form A.

FIG. 2 illustrates the DSC thermogram of armodafinil Form A.

FIG. 3 illustrates the XRD pattern of armodafinil hemihydrate prepared by the exposure of Form A to 100% relative humidity for 7 days at 30° C.

FIG. 4 illustrates the powder XRD pattern of armodafinil Form B.

FIG. 5 illustrates the powder XRD pattern of armodafinil Form C.

FIG. 6 illustrates the powder XRD pattern of armodafinil Form D.

FIG. 7 illustrates the powder XRD pattern of armodafinil Form E.

FIG. 8 illustrates the XRD patterns of dimethyl carbonate solvate, methyl acetate solvate, and THF solvate.

FIG. 9 illustrates the powder XRD pattern of armodafinil acetic acid solvate according to WO 2004/060858.

### DETAILED DESCRIPTION OF THE INVENTION

The invention encompasses crystalline forms of armodafinil and processes for making these crystalline forms of armodafinil. Each solid form possesses properties that are useful to the pharmaceutical formulator during formulation. These and other properties also may be advantageous to the process chemist when designing scale up synthesis, purification, and/or storage conditions of armodafinil. The processes described herein are also useful in the production of the crystalline forms of armodafinil in laboratory and commercial scale operations.

As used herein, the term “room temperature” refers to a temperature of about 10° C. to about 30° C.

As used herein, the term “reduced pressure” refers to a pressure of less than about 100 mmHg.

As used herein, the term “ambient pressure” refers to a pressure of less than about 760 mmHg.

As used herein, the term “water content” refers to the content of water based upon the Loss on Drying method (the “LOD” method) as described in Pharmacopeial Forum, Vol. 24, No. 1, p. 5438 (January-February 1998), the Karl Fisher assay for determining water content, or thermo gravimetric analysis (TGA). All percentages herein are by weight percent unless otherwise indicated.

Those skilled in the art will also understand that the term “monohydrate” when referring to armodafinil means that there is one water molecule for every molecule of armodafinil. In other words, monohydrate armodafinil has a water content of about 5-7% w/w. Similarly, those skilled in the art will also understand that the term “hemihydrate” when referring to armodafinil signifies that there is one molecule of water for every two molecules of armodafinil. Thus, hemihydrate armodafinil has a water content of about 3-4% w/w.

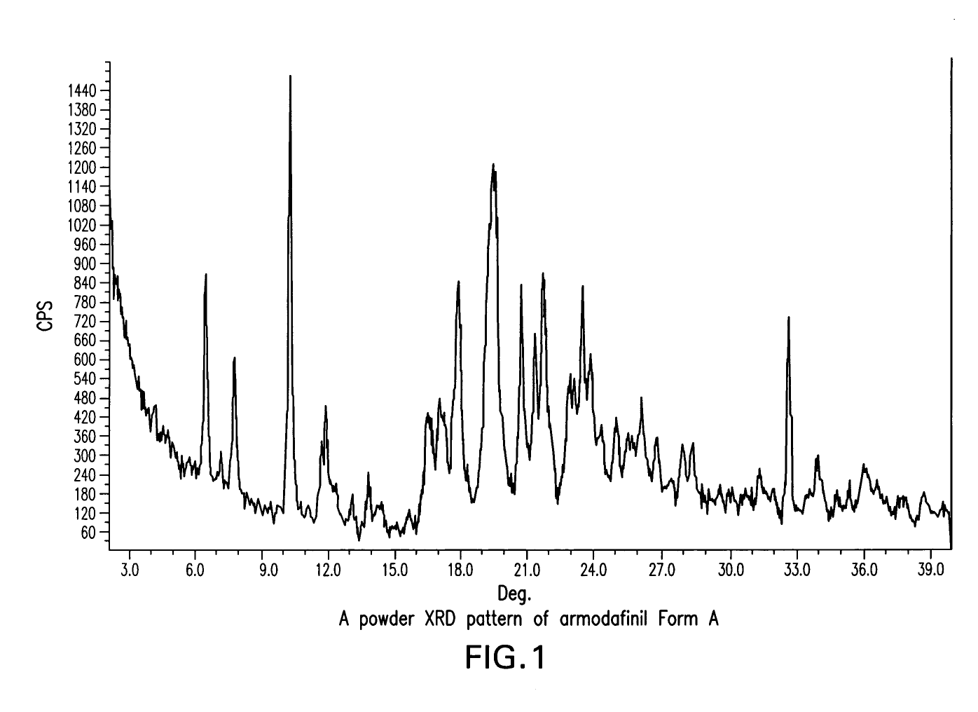

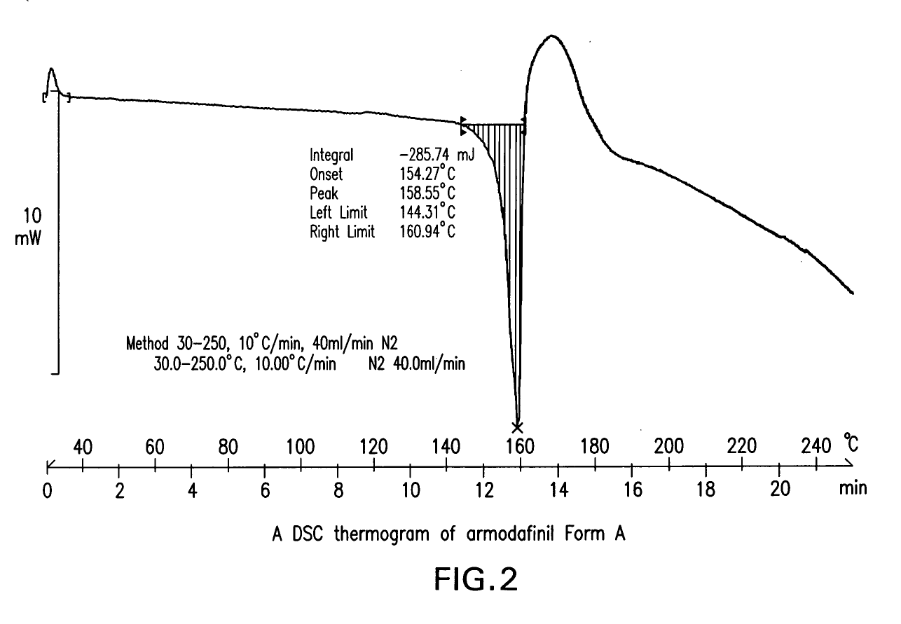

In one embodiment the invention encompasses a crystalline form of armodafinil characterized by a powder XRD pattern having peaks at about 6.5, 10.3, 17.9, 19.5 and 21.8±0.2 degrees 2-theta, herein defined as armodafinil Form A. Armodafinil Form A may be further characterized by at least one of the following: a powder XRD pattern having additional peaks at about 7.8, 20.8, 21.4, 23.5 and 32.6±0.2 degrees 2-theta substantially as depicted in FIG. 1; a DSC thermogram having an endothermic peak at about 154° C. substantially as depicted in FIG. 2; or a weight loss of about 0.3% at temperature of about 25° C. to about 120° C. as measured by TGA. The measured weight loss of 0.3% indicates armodafinil Form A may be considered to be anhydrous by those skilled in the art. Armodafinil Form A has a melting point of about 146° C. to about 147° C.

Form A is stable by pressure of about 2-10 tons which is used in the compression stage during the tablet preparation.

The invention provides a method of preparing armodafinil Form A by crystallizing it from acetonitrile. The process comprises: combining armodafinil with acetonitrile; heating; cooling; and isolating. After heating the combination of the armodafinil with acetonitrile, a solution or a slurry is obtained. Typically, armodafinil can be combined at a ratio of about 0.05 g/ml to about 0.15 g/ml of armodafinil to acetonitrile. The heating is preferably performed at a temperature of about 60° C. to about reflux temperature, more preferably, at a temperature of about 45° C. to 60° C. Preferably, the heating is for about 20 minutes to about 30 minutes. Preferably, the cooling is to about room temperature, more preferably, to a temperature of about 15° C. to about 30° C., most preferably, to a temperature of about 18° C. to about 25° C. Depending upon the particle size desired, during the cooling step, the solution or a slurry may be stirred. Preferably, the cooling is for about 3 hours to about 17 hours. For example, 2000 mg armodafinil is combined with 30 ml of acetonitrile and heated at reflux temperature for 1 hour. The obtained crystals of armodafinil Form A may be isolated by filtration. The crystals may be further dried. Preferably, the drying is by heating in a vacuum oven at 50° C. for 4 hours.

Another embodiment of the invention encompasses a crystalline form of armodafinil characterized by a powder XRD pattern having peaks at about 7.0, 9.3, 12.2, 14.4 and 16.2±0.2 degrees 2-theta, herein defined as armodafinil Form B. Armodafinil Form B may be further characterized by a powder XRD pattern having additional peaks at about 21.3, 23.3, 24.2, 24.7 and 25.1±0.2 degrees 2-theta or a powder XRD pattern substantially as depicted in FIG. 4. The crystalline form may be acetic acid solvate.

The invention further encompasses a method of preparing armodafinil Form B by crystallizing it from acetic acid. The process comprises: combining armodafinil with acetic acid; heating; cooling; and isolating. Preferably, the armodafinil is combined at a ratio of about 0.07 g/ml to about 0.12 g/ml of acetic acid. The heating is preferably at a temperature of about 70° C. to about reflux temperature, more preferably, at a temperature of about 75° C. Preferably, after heating a solution is obtained. The heating may be done for a time sufficient to form the desired dissolution of the armodafinil in the acetic acid. Preferably, the cooling is to a temperature of less than about 0° C., more preferably, to a temperature of about −5° C. to about −20° C. For example, 100 mg armodafinil is dissolved in 1 ml acetic acid and subsequently heated at reflux temperature for about 20 minutes. The solution is then cooled at −19° C. The obtained crystals of armodafinil Form B are isolated by decantation or filtration.

The present invention further provides a method of preparing armodafinil acetic acid solvate, as illustrated in FIG. 8, comprising drying armodafinil Form B. Preferably, the process comprises heating armodafinil Form B to a temperature of about 45° C. to about 55° C., preferably, to about 50° C. The method may be conducted at ambient or reduced pressure. The time required to obtain armodafinil acetic acid solvate may be vary depending upon other factors such as the drying temperatures. For example, the crystals of form B are heated at 50° C. in a vacuum oven for 4 hours to obtain armodafinil acetic acid solvate.

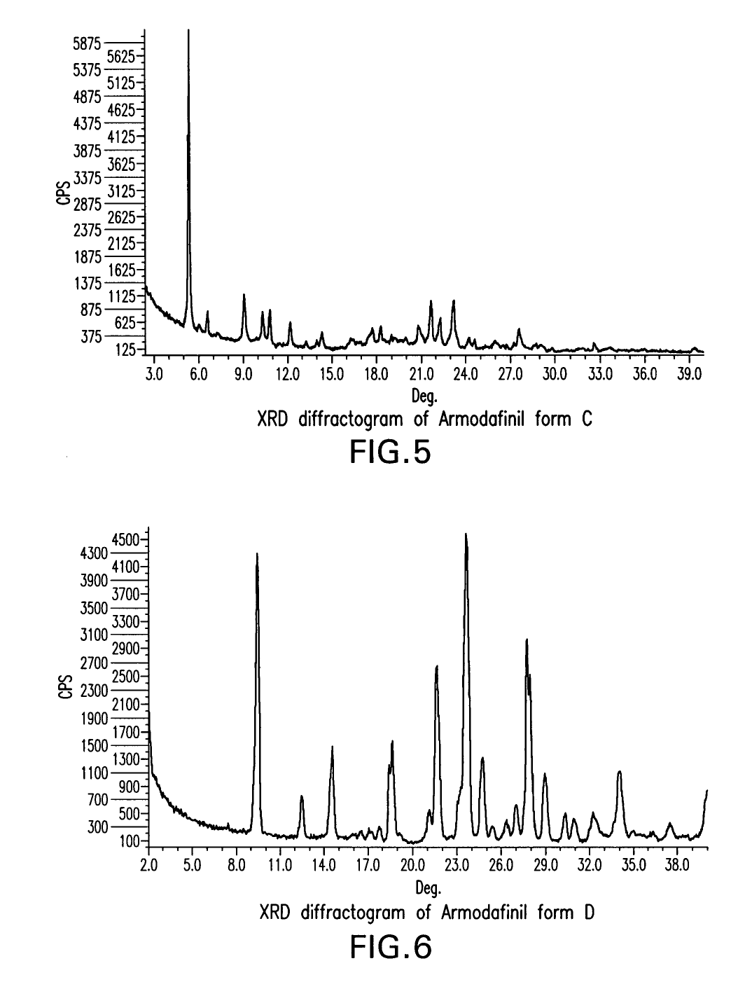

Another embodiment of the invention encompasses a crystalline form of armodafinil characterized by a powder XRD pattern having peaks at about 5.4, 9.1, 10.3 and 10.8±0.2 degrees 2-theta, herein defined as armodafinil Form C. Armodafinil Form C may be further characterized by a powder XRD pattern having peaks at about 12.2, 21.7, 22.3, 23.2 and 27.6±0.2 degrees 2-theta or a powder XRD pattern substantially as depicted in FIG. 5. The crystalline form may be dioxane solvate. A method of preparing armodafinil Form C comprises crystallizing it from dioxane. The process comprises: combining armodafinil with dioxane; heating; cooling and isolating. Preferably, armodafinil is combined at a ratio of about 0.04 g/ml to about 0.25 g/ml of dioxane. Preferably, the heating is to a temperature of about 55° C. to about reflux temperature, more preferably, to a temperature of about 65° C. Preferably, after heating a solution is obtained. For example, 35.5 mg armodafinil are combined with 0.8 ml dioxane and heated at 65° C. until the armodafinil is dissolved in the dioxane. Preferably, the cooling is to a temperature of less than about 0° C. Cooling the solution is more preferably to a temperature of about −5° C. to about −20° C., and most preferably at −19° C. The obtained crystals of armodafinil Form C are isolated by decantation or filtration.

Another embodiment of the invention encompasses a crystalline form of armodafinil characterized by a powder XRD pattern having peaks at about 9.4, 12.5, 14.5 and 18.6±0.2 degrees 2-theta, herein defined as armodafinil Form D. Armodafinil Form D may be further characterized by a powder XRD pattern having peaks at about 21.7, 23.7, 24.8, 27.8, 29.0 and 34.1±0.2 degrees 2-theta or a powder XRD substantially as depicted in FIG. 6. The crystalline form may be dimethyl carbonate solvate.

A method of preparing armodafinil Form D comprising crystallizing it from dimethyl carbonate. The process comprises: combining armodafinil with dimethyl carbonate; heating; cooling; and isolating. Preferably, armodafinil is combined at a ratio of about 0.1 g/ml to about 0.2 g/ml of dimethyl carbonate. Preferably, the heating is to a temperature of about 55° C. to about reflux temperature, more preferably, to a temperature of about 60° C. to about 70° C., most preferably, to a temperature of about 65° C. Preferably, after heating a solution is obtained. Optionally, water may be added to the dimethyl carbonate. Preferably, the water and the dimethyl carbonate are at a ratio of about 0.95:1.15, more preferably, 1:1 by volume. For example, 47.1 mg armodafinil are combined with 0.4 ml dimethyl carbonate and 0.4 ml water and heated at a temperature of 65° C. until the armodafinil dissolves. Preferably, the cooling is to a temperature of about 15° C. to about 30° C., more preferably, to a temperature of about 18° C. to about 25° C. Depending upon the particle size desired, the solution may optionally be stirred during the cooling step. Preferably, the cooling is for about 3 hours to about 17 hours. The obtained crystals of armodafinil Form D are isolated by decantation or filtration.

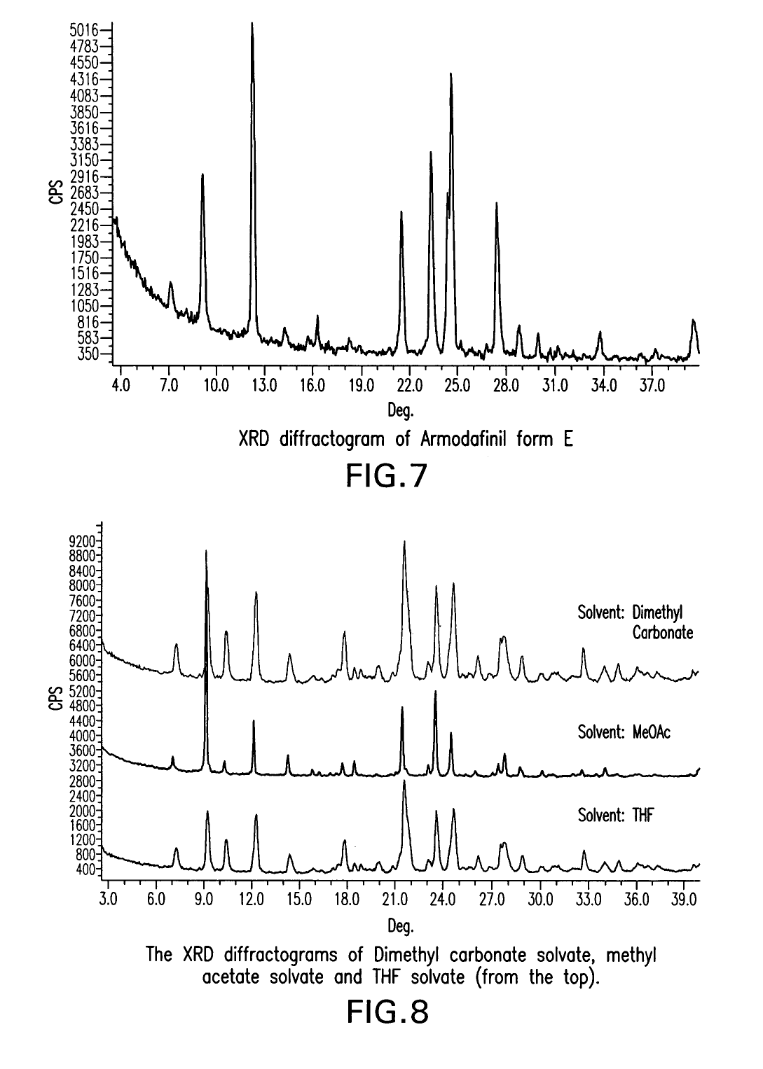

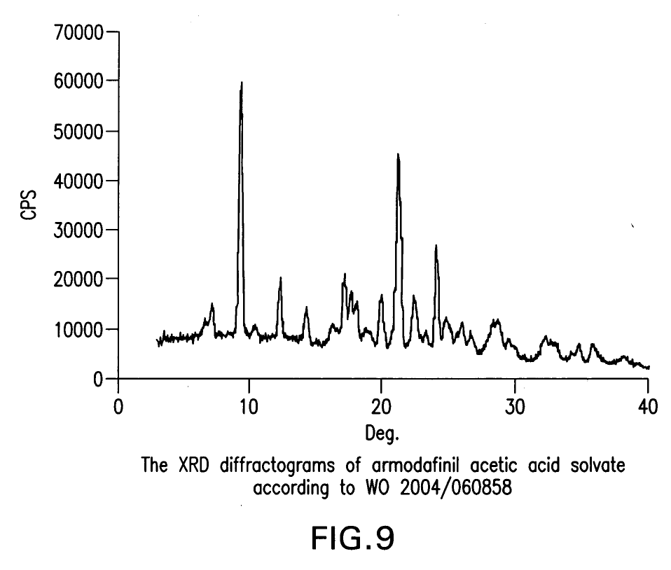

Another embodiment of the invention encompasses a crystalline form of armodafinil characterized by a powder XRD pattern having peaks at about 7.1, 9.1, 12.2 and 16.3±0.2 degrees 2-theta, herein defined as armodafinil Form E. Armodafinil Form E may be further characterized by a powder XRD pattern having peaks at about 24.4, 24.6, 27.4, 28.8, 29.9 and 33.8±0.2 degrees 2-theta or a XRD pattern as depicted in FIG. 7. The crystalline form may be methylethylketone solvate.

The invention further provides a method of preparing armodafinil Form E by crystallizing it from methylethylketone. The process comprises: combining armodafinil with methylethylketone; heating; cooling; and isolating. Preferably, armodafinil is combined at a ratio of about 0.02 g/ml to about 0.2 g/ml of methylethylketone. Preferably, the heating is to a temperature of about 35° C. to about 45° C., more preferably, to a temperature of about 40° C. For instance, an amount of about 0.2 g of armodafinil may be heated about 16 hours. For example, 18.3 mg armodafinil is combined in a slurry with 1 ml methylethylketone and heated at 40° C. for 16 hours. Preferably, the cooling is to a temperature of about 5° C. to about −5° C., more preferably, to a temperature of about 0° C. The obtained crystals of armodafinil Form E may be isolated by decantation or filtration.

The invention also encompasses methods for preparing armodafinil Form I by drying or humidifying the novel crystalline forms described above.

One method for preparing armodafinil Form I comprises drying armodafinil Form C. Preferably, the drying comprises heating armodafinil Form C to a temperature of about 45° C. to about 55° for at least 4 hours. More preferably, the heating is to a temperature of about 50°. The method may be conducted at ambient or reduced pressure. The time will depend upon the amount of material to dry; for example, typically about 0.04 g of crystalline armodafinil Form C is dried for about 4 hours to obtain armodafinil Form I.

Another method for preparing armodafinil Form I comprises drying armodafinil Form D. The process can be stopped prematurely to obtain a mixture. Preferably, the drying comprises heating armodafinil Form D to a temperature of about 50° C. at ambient or reduced pressure for a time sufficient to form armodafinil Form I. The time will depend upon the amount of armodafinil Form D; for example, typically about 0.05 g of crystalline armodafinil Form D is dried for about 4 hours.

Another method for preparing armodafinil Form I comprises exposing armodafinil Form A to 100% relative humidity at a temperature of about 20° C. to about 40° C., preferably at a temperature of about 30° C. to about 40° C., for a time sufficient to form armodafinil Form I. Preferably, the exposure is over a period of about 7 days to about 14 days, more preferably, 7 days. The time will depend upon the amount of armodafinil Form A; for example, typically about 200 mg of armodafinil Form A is exposed to 100% relative humidity for 7 days to obtain armodafinil Form I.

Armodafinil Form I may also be prepared by drying armodafinil Form IV. Preferably, the process comprises heating armodafinil Form IV to a temperature of about 120° C. to 150° C. for at least about 10 minutes. Preferably, the heating is to a temperature of about 145° C. Preferably, the heating is conducted over a period of about 15 minutes about 30 minutes. For example, armodafinil Form IV 200 mg is heated for about 10 minutes. Form IV may be prepared according to WO 2004/060858.

A further method of preparing armodafinil Form I comprises submitting armodafinil Form IV to a pressure of about 2 tons to about 10 tons to obtain armodafinil Form I. Preferably, the pressure is applied for a period of about 1 minute to about 5 minutes. The time will depend upon the amount of armodafinil Form IV; for example, armodafinil 100 mg of Form IV is pressed with 10 tons for 1 minute.

Form IV is characterized by a weight loss that is 0.3% at the range of about 25° C. to about 100° C. as measured by TGA.

Another method for preparing armodafinil Form I comprises grinding armodafinil Form IV. Preferably, the grinding is carried out over a period of about 1 minute to about 5 minutes, more preferably, about 1 min. Depending on the amount of armodafinil Form IV, different grinding methods may be used. For example, 200 mg of armodafinil Form IV is ground with a mortar and pestle for 1 minute.

The invention encompasses armodafinil THF solvate.

The invention also encompasses armodafinil THF solvate characterized by having PXRD peaks at 7.3, 9.3, 10.4, 12.3, 14.4, 17.8, 21.6, 23.6, 24.7±0.2 degrees 2-theta. The THF solvate may be characterized by a weight loss that is 39% at the range of about 25° C. to about 100° C. as measured by TGA.

In another embodiment the invention encompasses a method of preparing armodafinil THF solvate comprising crystallizing it from THF. The process comprises: combining armodafinil with THF; heating; cooling; and isolating. Preferably, armodafinil is combined at a ratio of about 0.1 g/ml to about 0.2 g/ml of THF. Preferably, the heating is to a temperature of about 55° C. to about reflux temperature, more preferably, to a temperature of about 65° C. Preferably, after heating, a solution is obtained. Preferably, the cooling is to about room temperature, more preferably, to a temperature of about 15° C. to about 30° C., most preferably, to a temperature of about 18° C. to about 25° C. Preferably, the cooling is for at least 3 hours. Depending upon the desired particle size, the solution may be stirred while cooling. For example, 2 g of armodafinil is combined with 40 ml of THF and heated at reflux temperature for 1 hour forming a solution. The solution is then stirred while being cooled at room temperature for 3 hours which resulted in the formation of crystals of armodafinil THF solvate. The crystals of armodafinil THF solvate may be isolated by filtration or decantation.

The invention encompasses arnodafinil methyl acetate solvate.

The invention also encompasses armodafinil methyl acetate solvate characterized by having PXRD peaks at 7.3, 9.3, 10.4, 12.3, 14.4, 17.8, 21.6, 23.6, 24.7±0.2 degrees 2-theta.

Another embodiment of the invention encompasses a method of preparing armodafinil methyl acetate solvate comprising crystallizing it from methyl acetate. The process comprises: combining armodafinil with methyl acetate; heating; cooling; and isolating. Preferably, armodafinil is combined at a ratio of about 0.02 g/ml to about 0.5 g/ml of methyl acetate. Preferably, the heating is to a temperature of about 45° C. to about 55° C., more preferably, about 50° C. to about reflux temperature. Preferably, after heating, a solution is obtained. Preferably, the cooling is to about room temperature, more preferably, to a temperature of about 15° C. to about 30° C., most preferably, to a temperature of about 18° C. to about 25° C. Preferably, the cooling is for about 12 hours to about 48 hours, more preferably, for about 15 hours to about 28 hours. Optionally, further cooling step is performed, preferably, to a temperature of about −5° C. to about −20° C. Depending on the size of particles desired, the solution may be stirred during cooling. For example, 20.5 mg armodafinil is combined with 1 ml methyl acetate and is heated to a temperature of 45° C. to about 55° C., more preferably, about 50° C., for 1 hour, forming a solution. The solution is then stirred while cooling at room temperature for 2 days. After 2 days the solution is cooled at −19° C. which caused the formation of armodafinil methyl acetate solvate. The obtained crystals of armodafinil methyl acetate solvate may be done by isolated by decantation or filtration.

Another embodiment of the invention encompasses a method for preparing armodafinil Form IV comprising drying armodafinil THF solvate. Preferably, the process comprises: heating armodafinil THF solvate to a temperature of about 45° C. to about 55° C., more preferably, about 50° C., for a time sufficient to form armodafinil Form IV. The heating may occur at ambient or reduced pressure. For example, armodafinil THF solvate is heated at a temperature of 50° C. in a vacuum oven under reduced pressure for 4 hours to obtain armodafinil Form IV.

The invention further encompasses a method of preparing armodafinil Form IV comprising drying armodafinil Form E. Preferably, the drying comprises heating the armodafinil Form E to a temperature of about 45° C. to about 55° C., more preferably, about 50° C. The drying may occur at under at ambient pressure or under a vacuum for a time sufficient to form armodafinil Form IV. The time will be determined upon the amount of starting armodafinil Form E. For example, about 0.2 g of armodafinil is dried for about 4 hours under vacuum. For example, armodafinil Form E is heated at 50° C. in a vacuum oven for 4 hours to obtain armodafinil Form IV.

A further process for preparing armodafinil Form IV comprises drying armodafinil methyl acetate solvate. Preferably, the process comprises: heating armodafinil methyl acetate solvate at a temperature above 45° C. to about 55° C., more preferably, about 50° C., for a time sufficient to form crystals of armodafinil Form IV. The heating may occur at ambient or reduced pressure. For example, armodafinil methyl acetate solvate is heated at a temperature of 50° C. in a vacuum oven under reduced pressure for 4 hours to obtain armodafinil Form IV.

Another embodiment of the invention encompasses armodafinil in hydrate form.

The invention encompasses armodafinil hemihydrate.

The invention also encompasses armodafinil hemihydrate characterized by PXRD peaks at 6.8, 10.5, 13.5, 14.2, 19.2, 20.2, 21.1, 22.5, 23.7, 26.2±0.2 degrees 2-theta.

The invention also encompasses a process for preparing armodafinil hemihydrate, comprising exposing armodafinil Form A to 100% humidity for at least about 7 days at a temperature of about 20° C. to about 40° C. Preferably, the temperature is about 30° C.

Another embodiment of the invention encompasses armodafinil monohydrate. Armodafinil monohydrate may be prepared by a method comprising exposing armodafinil Form 1 to 100% relative humidity at a temperature of about 30° C. to about 60° C. for at least 7 days. Preferably, the heating is to a temperature of about 30° C. to about 40° C. In one example 200 mg of armodafinil Form I is heated at 30° C. in 100% relative humidity for 13 days.

A further embodiment of the invention encompasses a pharmaceutical formulation comprising a therapeutically effective amount of at least one of armodafinil Forms A, B, C, D, E, THF solvate, methyl acetate solvate, hemihydrate or monohydrate, preferably Form A, and at least one pharmaceutically acceptable excipient. Also provided is a process for preparing such pharmaceutical compositions by mixing the crystalline forms with a pharmaceutically acceptable carrier. The pharmaceutical compositions can be used for treatment of excessive sleepiness.

Pharmaceutically acceptable excipients may include excipients commonly used in pharmaceutical formulations. Pharmaceutically acceptable excipients used in the formulation include, but are not limited to, diluents, binders, disintegrants, lubricants, flavorings, sweeteners, or preservatives.

Diluents used in the formulation include diluents commonly used in pharmaceutical formulations. For example, diluents include, but are not limited to, cellulose-derived materials, such as powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents, such as calcium carbonate and calcium diphosphate; waxes; sugars; sugar alcohols, such as mannitol and sorbitol; acrylate polymers and copolymers; pectin; dextrin; or gelatin.

Binders used in the formulation include binders commonly used in pharmaceutical formulations. For example, binders include, but are not limited to, acacia gum, pregelatinized starch, sodium alginate, or glucose.

Disintegrants used in the formulation include disintegrants commonly used in pharmaceutical formulations. For example, disintegrants include, but are not limited to, sodium starch glycolate, crospovidone, or low-substituted hydroxypropyl cellulose.

Lubricants used in the formulation include lubricants commonly used in pharmaceutical formulations. For example, lubricants include, but are not limited to magnesium stearate, calcium stearate, or sodium stearyl fumarate.

The pharmaceutical formulations of the invention may be provided in dosage forms for oral, parenteral (including subcutaneous, intramuscular, and intravenous), inhalant, or ophthalmic administration. Although the most suitable route in any given case will depend on the nature and severity of the condition being treated, the most preferred route of the invention is oral. Dosages may be conveniently presented in unit dosage form and prepared by any of the methods well-known in the art of pharmacy.

Dosage forms include solid dosage forms, such as tablets, powders, capsules, suppositories, sachets, troches, and lozenges, as well as liquid suspensions and elixirs.

Capsule dosages will contain the solid composition within a capsule which may be made of gelatin or other conventional encapsulating material.

Tablets and powders may be coated, for example, with an enteric coating. The enteric-coated powder forms may have coatings comprising phthalic acid cellulose acetate, hydroxypropylmethyl-cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

Another embodiment of the invention encompasses methods of treating patients suffering from excessive sleepiness associated with narcolepsy, shift work sleep disorder (“SWSD”), and obstructive sleep apnea/hypopnea syndrome (“OSA/HS”) comprising administering to a patient in need thereof a therapeutically effective amount of a pharmaceutical formulation comprising at least one armodafinil forms of the invention and at least one pharmaceutically acceptable excipient.

Having described the invention with reference to certain preferred embodiments, other embodiments will become apparent to one skilled in the art from consideration of the specification. The invention is further defined by reference to the following examples describing in detail the preparation of armodafinil crystalline forms of the invention. It will be apparent to those skilled in the art that many modifications, both to materials and methods, may be practiced without departing from the scope of the invention.

### EXAMPLES

### PXRD

X-Ray powder diffraction data were obtained by using methods known in the art. A SCINTAG powder X-ray diffractometer model X'TRA equipped with a solid-state detector was employed with copper radiation of 1.5418 Å and a round aluminum sample holder with zero background. The scanning parameters included: range: 2-40 degrees two-theta; scan mode: continuous scan; step size: 0.05 deg.; and a rate of 5 deg/min. All peak positions are within ±0.2 degrees two theta.

One skilled in the art will appreciate that there is a small amount of uncertainty involved in PXRD measurements, generally of the order of about +0.2 degrees 2-theta for each peak. Accordingly, PXRD peak data herein are presented in the form of “a PXRD pattern having peaks at A, B, C, etc. ±0.2 degrees 2-theta.” This indicates that, for the crystalline form in question, the peak at A could, in a given instrument on a given run, appear somewhere between A ±0.2 degrees 2-theta, the peak at B could appear at B ±0.2 degrees 2-theta, etc. Such small, unavoidable uncertainty in the identification of individual peaks does not translate into uncertainty with respect to identifying individual crystalline forms since it is generally the particular combination of peaks within the specified ranges, not any one particular peak, that serves to unambiguously identify crystalline forms.

### DSC

DSC analysis was performed using a Mettler 821 Stare. Samples of about 4 mg were analyzed in standard 40 μl alumina crucibles covered by lids with 3 holes. Each sample was scanned at a rate of 10° C./min from 25° C. to 200° C. The oven was constantly purged with nitrogen gas at a flow rate of 40 ml/min.

### TGA

TGA analysis was performed using a Metler M3 thermo gravimeter. Samples of about 8 mg were analyzed in standard 150 μl alumina crucibles covered by lids with 1 hole. Each sample was scanned at a rate of 10° C./min from 25° C. to 200° C. A blank was subtracted from the sample. The oven was constantly purged with nitrogen gas at a flow rate of 40 ml/min.

### Example 1

### Preparation of Armodafinil Form A

Armodafinil (2 g) was combined with 30 ml acetonitrile and heated at reflux temperature for 1 hour and was then cooled to room temperature. This resulted in the formation of crystals. The crystals were collected by filtration and heated in a vacuum oven at 50° C. for 4 hours. An XRD analysis showed the product to be armodafinil Form A.

### Example 2

### Preparation of Armodafinil Form A

Armodafinil (100 mg) was combined with 1 ml acetonitrile and heated at reflux temperature for 20 minutes and then stirred while cooling at room temperature overnight. This resulted in the formation of crystals. The crystals were collected by filtration and heated in a vacuum oven at 50° C. for 4 hours. An XRD analysis showed the product to be armodafinil Form A.

### Example 3

### Preparation of Armodafinil Hemihydrate

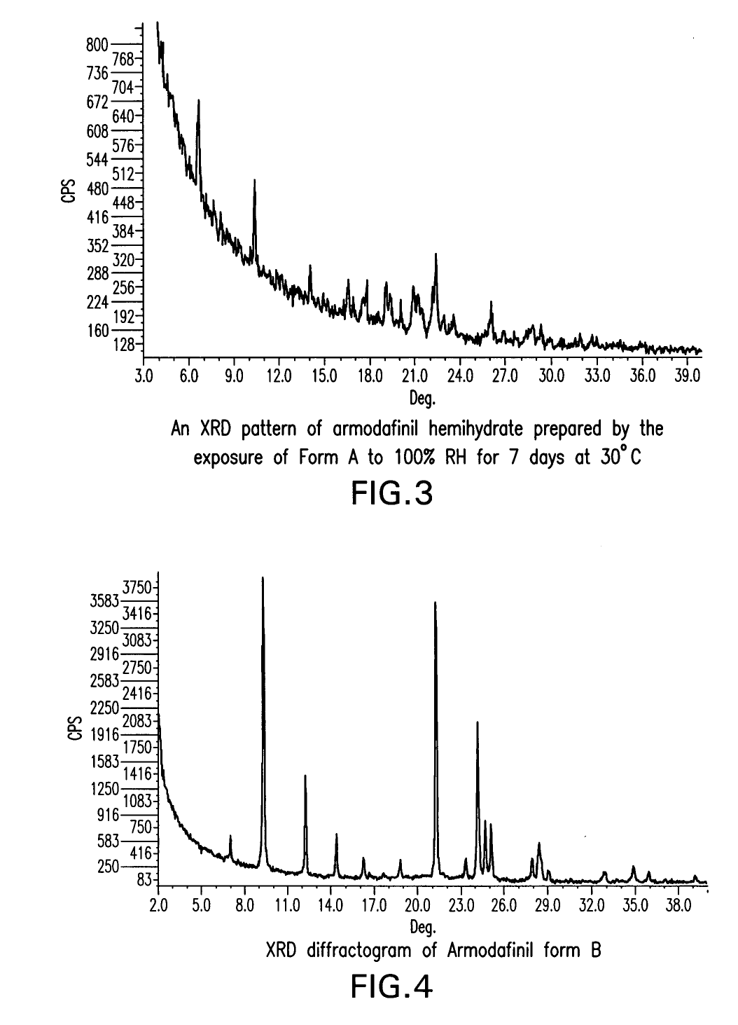

Armodafinil Form A (200 mg) was exposed to 100% humidity for 7 days at 30° C. Under these conditions, armodafinil Form A transformed to armodafinil Form I, as shown in the table below. The XRD diffractogram of armodafinil Form I is illustrated in FIG. 3.

| | % Weight loss | Crystal form |
|---|---|---|
| RH % | (by TGA) | (by XRD) |
| Initial | 0.3 | Form A |
| 100 | 3.5 | Form I |

### Example 4

### Preparation of Armodafinil Form B and the Acetic Acid Solvate of Armodafinil

Armodafinil (100 g) was combined with 1 ml acetic acid and heated at 75° C. for 20 minutes forming a solution. The solution was then cooled to −19° C. which resulted in the formation of crystals. The crystals were subsequently collected by decantation. The crystals were analyzed by XRD and was identified as armodafinil Form B. The crystals were then heated at 50° C. in a vacuum oven for 4 hours and analyzed by XRD. The XRD showed the presence of acetic acid solvate.

### Example 5

### Preparation of Armodafinil Form C and Armodafinil Form I

Armodafinil (35.5 mg) was combined with 0.8 ml dioxane and heated at 65° C. overnight. Complete dissolution was observed. The solution was cooled at −19° C. which resulted in the formation of crystals. The crystals were subsequently collected by decantation.

The crystals were analyzed by XRD and were identified as armodafinil Form C. The crystals were then heated at 50° C. in a vacuum oven for 4 hours and analyzed by XRD. The XRD showed the presence of armodafinil Form I.

### Example 6

### Preparation of Armodafinil Form D

Armodafinil (47.1 mg) was combined with 0.4 ml dimethyl carbonate and 0.4 ml water and was heated to 65° C., and thereafter cooled to room temperature (the heating a cooling was repeated twice). The solution was stirred at room temperature overnight which resulted in the formation of crystals. The crystals were subsequently collected by decantation.

The crystals were analyzed by XRD and identified as armodafinil Form D. The crystals were then heated at 50° C. in a vacuum oven for 4 hours and analyzed by XRD. The XRD showed the presence of armodafinil Form I.

### Example 7

### Preparation of Armodafinil Form D

Armodafinil (100 g) was combined with 1 ml dimethyl carbonate and was heated at 65° C. for 20 minutes forming a solution. The solution was then stirred while cooling at room temperature overnight which resulted in the formation of crystals. The crystals were recovered by decantation and were identified as armodafinil Form D by XRD.

### Example 8

### Preparation of Armodafinil Form E and Armodafinil Form Iv

Armodafinil (18.3 mg) was mixed with 1 ml methylethylketone and heated at 40° C. for 16 hours forming a solution. The solution was then cooled to 0° C. which caused the formation of crystals. The crystals were subsequently collected by decantation.

The crystals were analyzed by XRD and identified as armodafinil Form E. The crystals were then heated at 50° C. in a vacuum oven for 4 hours and analyzed by XRD. The XRD showed the presence of armodafinil Form IV.

### Example 9

### Preparations of Armodafinil Form I from Armodafinil Form Iv

Armodafinil Form IV (200 mg) was heated at 145° C. for 10 min forming a heated sample. Analysis by XRD of the heated sample showed the presence of armodafinil Form I.

### Example 10

### Preparations of Armodafinil Form I from Armodafinil Form Iv

Armodafinil Form IV (100 mg) was pressed with pressure of 2 tons or 10 tons for 1 min forming a pressed sample. Analysis by XRD of the pressed sample showed the presence of armodafinil Form I.

### Example 11

### Preparations of armodafinil Form I from Armodafinil Form IV

Armodafinil Form IV (100 mg) was ground with mortar and pestle for a few minutes forming a ground sample. Analysis by XRD of the pressed sample showed the presence of armodafinil Form I.

### Example 12

### Preparation of THF Solvate and Armodafinil Form Iv

Armodafinil (2 g) was combined with 40 ml THF and heated at reflux temperature for 1 hour forming a solution. The solution was then stirred while cooling at room temperature for 3 hours resulting in the formation of crystals. The crystals were subsequently collected by filtration.

The crystals were analyzed by XRD and identified as THF solvate. The crystals were then heated at 50° C. in a vacuum oven for 4 hours and analyzed by XRD. The XRD showed the presence of armodafinil Form IV.

### Example 13

### Preparation of THF Solvate and Armodafinil Form Iv

Armodafinil (100 mg) was combined with 1 ml THF and heated at 65° C. for 20 minutes forming a solution. The solution was then stirred while cooling at room temperature overnight which resulted in the formation of crystals. The crystals were subsequently collected by decantation.

The crystals were analyzed by XRD and identified as THF solvate. The crystals were then heated at 50° C. in a vacuum oven for 4 hours and analyzed by XRD. The XRD showed the presence of armodafinil Form IV.

### Example 14

### Preparation of Methyl Acetate Solvate and Armodafinil Form Iv

Armodafinil (20.5 mg) was combined with 1 ml methyl acetate and was heated at 50° C. for 60 minutes forming a solution. The solution was then stirred while cooling at room temperature for 2 days. The solution was then further cooled to −19° C. which resulted in the formation of crystals. The crystals were subsequently recovered by decantation.

The crystals were analyzed by XRD identified as methyl acetate solvate. The crystals were then heated at 50° C. in a vacuum oven for 4 hours and analyzed by XRD. The XRD showed the presence of armodafinil Form IV.

### Example 15

### Preparation of Armodafinil Monohydrate

TABLE 1
TGA and XRD results for armodafinil Form I exposed to
100% Relative Humidity at 30° C. for 13 days

| | % Weight loss | Crystal form |
|---|---|---|
| % RH | (by TGA) | (by XRD) |
| 100 | 5.6 | Form I |
| Initial | 0.1 | Form I |

TABLE 2
TGA and XRD results for armodafinil Form I exposed to
100% Relative Humidity at 60° C. for 13 days

| | % Weight loss | Crystal form |
|---|---|---|
| % RH | (by TGA) | (by XRD) |
| 100 | 7.4 | Form I |
| Initial | 0.1 | Form I |

1-59. (canceled)

60. A process for preparing armodafinil Form I comprising drying or humidifying at least one armodafinil crystalline forms selected from the group consisting of Form A, Form B, Form C, Form D, Form E, and crystalline armodafinil acetic acid.

61. The process of claim 60, wherein the process comprises drying armodafinil Form C.

62. The process of claim 61, wherein the drying comprises heating armodafinil Form C is to a temperature of about 45° C. to about 55° C.

63. The process of claim 61, wherein the heating is at a temperature of about 50° C.

64. A process for preparing armodafinil Form I comprising drying armodafinil Form D.

65. The process of claim 64 wherein the drying comprises heating armodafinil Form D to a temperature of about 50° C.

66. A process for preparing armodafinil Form I comprising exposing armodafinil Form A to 100% relative humidity at a temperature of about 20° C. to about 40° C.

67. The process of claim 66 wherein the exposure is over a period of about 7 days to about 14 days.

68. A process for preparing armodafinil Form I comprising drying armodafinil Form IV.

69. The process of claim 68 wherein drying comprises heating armodafinil Form IV to a temperature of about 120° C. to 150° C.

70. A process for preparing armodafinil Form I comprising submitting armodafinil Form IV to a pressure of about 2 tons to about 10 tons to obtain armodafinil Form I.

71. (canceled)

72. A process for preparing armodafinil Form I comprising grinding armodafinil Form IV.

73-107. (canceled)
